(19) United States
(12) Patent Application Publication
(10) Pub. No.: US 2023/0109311 A1
(43) Pub. Date: Apr. 6, 2023

(54) HERBAL FORMULATION AND METHOD OF ADMINISTERING THE SAME

(71) Applicant: Robert R. Beltran, Rancho Santa Fe, CA (US)

(72) Inventor: Robert R. Beltran, Rancho Santa Fe, CA (US)

(21) Appl. No.: 17/494,598

(22) Filed: Oct. 5, 2021

(51) Int. Cl. A61K 36/889; A61K 36/185; A61K 36/67; A61K 36/734; A61K 36/258; A61K 36/16; A61K 36/15; A61K 36/45; A61K 36/31; A61K 36/42

(52) U.S. Cl. CPC A61K 36/889; A61K 36/185; A61K 36/67; A61K 36/734; A61K 36/258; A61K 36/16; A61K 36/15; A61K 36/45; A61K 36/31; A61K 36/42

(57) ABSTRACT

The present disclosure relates to an herbal composition and a method of administering the composition including administering the composition in pill or capsule form on a routine basis and administering tadalafil on a routine basis in some instances for the purpose of increasing energy, alertness, clarity, memory, and sexual enhancement in individuals, and particularly in males.

### TECHNICAL FIELD

The embodiments generally relate to the field of herbal formulations and methods of administering herbal formulations.

### BACKGROUND

Formulations and methods of administering formulations designed for increased energy, alertness, mental capacity, and sexual enhancement may include a variety of undesirable side effects such as shakiness, jitters, lethargy, nausea, high blood pressure, low blood pressure, headache, upset stomach, back pain, muscle pain, stuffy nose, flushing, or dizziness and the like. The present disclosure relates to a composition of an herbal enhancement formulation and a method of administering said composition that may enhance energy while reducing undesirable side effects. The composition may provide enhanced mental alertness, clarity, and memory. The composition may provide a sense of well-being and contentment in addition to sexual enhancement, particularly in males.

### SUMMARY OF THE INVENTION

This summary is provided to introduce a variety of concepts in a simplified form that is further disclosed in the detailed description of the embodiments. This summary is not intended to identify key or essential inventive concepts of the claimed subject matter, nor is it intended for determining the scope of the claimed subject matter.

The present disclosure relates to a composition containing a number of components including, but not limited to, saw palmetto extract; damiana leaf extract; Piper nigrum fruit extract; hawthorn berry; muira paula bark 4:1 extract; Korean red ginseng; Catuaba bark 4:1 extract; Epimedium sagittatum leaf; Ginkgo biloba leaf; cuscata 4:1 seed extract; Tribulus terrestris vine; butea superba root; maca root; pine bark extract; cranberry extract; Avena sativa extract seed; sarsaparilla root; swedish flower; pumpkin seed; cayenne pepper; di-tocopheral acetate; zinc aspartate; L-carnitine; L-arginine HCl; and L-lysine.

The present disclosure relates to various methods of administering the composition including administering the composition in pill or capsule form on a routine basis, administering cannabis indica on a routine basis, and administering a vasodilator such as, but not limited to, tadalafil on a routine basis for the purpose of increasing energy, alertness, clarity, memory, and sexual enhancement in individuals, and particularly in males.

Both the foregoing general description and the following detailed description present embodiments intended to provide an overview or framework for understanding the nature and character of the embodiments disclosed herein. Any accompanying drawings are included to provide further understanding and are incorporated into and constitute a part of this specification. The drawings illustrate various embodiments of the disclosure, and together with the description explain the principles and operations thereof.

### BRIEF DESCRIPTION OF THE DRAWINGS

The features and advantages of the present disclosure will be more fully described in, or rendered obvious by the following detailed description of the preferred embodiments, which are to be considered together with the accompanying drawings wherein like numbers refer to like parts and further, wherein:

FIG. 1 depicts a flowchart of a method of administering a composition, in accordance with some embodiments described herein; and

FIG. 2 depicts observed enhancement in key areas in participants receiving administration of the method, in accordance with some embodiments described herein.

### DETAILED DESCRIPTION

The specific details of single embodiments or variations described herein are to the described composition and methods of use. Any specific details of the embodiments are used for demonstration purposes only and no unnecessary limitations or inferences are to be understood from there.

The present disclosure discloses a composition which may comprise, in a mixture, saw palmetto extract; damiana leaf extract; Piper nigrum fruit extract; hawthorn berry; muira paula bark 4:1 extract; Korean red ginseng; Catuaba bark 4:1 extract; Epimedium sagittatum leaf; Ginkgo biloba leaf; cuscata 4:1 seed extract; Tribulus terrestris vine; butea superba root; maca root; pine bark extract; cranberry extract; Avena sativa extract seed; sarsaparilla root; swedish flower; pumpkin seed; cayenne pepper; di-tocopheral acetate; zinc aspartate; L-carnitine; L-arginine HCl; and L-lysine.

The compositions disclosed may be encapsulated in a pill or capsule form according to conventional means. According to some embodiments, the composition is ingested by a user at least once a day and in some embodiments, the composition is ingested up to four times daily. The composition, as best seen below in Tables 1, 2, and 3, may increase blood flow to the genital area including increased blood flow into the corpus cavernosum to produce increased erectile strength induration in addition to healthier tissue. The composition may have a direct effect on the nerve endings of the genital area to increase sensitivity. The composition may increase free testosterone in the bloodstream and may increase blood flow to the brain including areas of the brain active during sexual activity and facilitate the increased release of pertinent neurotransmitters such as oxytocin, serotonin, and dopamine. The composition may have a direct effect on the pleasure areas of the brain and then in this manner may directly stimulate the release of neurotransmitters related to sexual activity.

According to some variations, a method of sexual enhancement may include administering the composition in four approximately equal doses throughout a twenty-four-hour period, administering cannabis indica daily, preferably prior to sleep, and/or administering tadalafil approximately every five days routinely.

In some embodiments the addition of cannabis indica, including THC, may activate cannabinoid receptors in the brain's reward system resulting in the stimulation and response to healthy pleasurable behaviors such as sexual activity. Additionally, THC may cause a surge of dopamine release to further stimulate pleasure. The combination of THC and the disclosed composition may be synergistic in that the composition may increase blood flow to the entire brain to stimulate the pleasure and erogenous areas of the brain, which may be augmented by the presence of THC.

In some embodiments the addition of tadalafil may relax smooth muscle within the body and increase blood flow to the corpus cavernosum. The addition of tadalafil may be synergistic with the ingested composition in that both components may increase blood flow to result in an additive effect.

According to various embodiments illustrating the practice of the present method and composition, a person having ordinary skill in the art will understand that the method and composition may be practiced or applied according to other variant embodiments. Many other possible modifications and variations of the details of the present composition and method based on different outlooks and applications may be made without departing from the spirit of this disclosure.

As best shown in Table 1, the composition may include various ingredients in a range of effective amounts according to the disclosed method.

TABLE 1

| Component | mg | IU |
|---|---|---|
| Saw Palmetto extract | 360-420 | — |
| Damiana Leaf extract | 360-420 | — |
| Piper Nigrum Fruit Extract | 15-25 | — |
| Hawthorn Berry | 440-540 | — |
| Muira Paula Bark 4:1 extract | 250-350 | — |
| Korean Red Ginseng | 440-560 | — |
| Catuaba Bark 4:1 extract | 360-440 | — |
| Epimedium Saggatatum leaf | 180-250 | — |
| Ginkgo Biloba Leaf | 400-600 | — |
| Cuscuta 4:1 See Extract | 60-120 | — |
| Tribulus Terrestris Vine | 330-425 | — |
| Butea Superba Root | 400-540 | — |
| Maca Root | 340-440 | — |
| Pine Bark extract | 260-340 | — |
| Cranberry extract | 60-100 | — |
| Avena Sativa extract seed | 40-100 | — |
| Sarsaparilla root | 60-120 | — |
| Swedish flower (pollen) | 40-80 | — |
| Pumpkin seed | 20-60 | — |
| Cayenne pepper | 600-1000 | — |
| Vitamin E (ditocopheral acetate) | — | 40-80 |
| Zinc Aspartate (20% elemental Zinc) | 30-70 | — |
| L-Carnitine | 400-540 | — |
| L-Arginine HCL | 360-440 | — |
| L-Lysine | 220-280 | — |

As best shown in Table 2, the composition may include various ingredients in a range of effective amounts according to the disclosed method.

TABLE 2

| Component | mg | IU |
|---|---|---|
| Saw Palmetto extract | 400 | — |
| Damiana Leaf extract | 400 | — |
| Piper Nigrum Fruit Extract | 20 | — |
| Hawthorn Berry | 480 | — |
| Muira Paula Bark 4:1 extract | 300 | — |
| Korean Red Ginseng | 500 | — |
| Catuaba Bark 4:1 extract | 400 | — |
| Epimedium Saggatatum leaf | 220 | — |
| Ginkgo Biloba Leaf | 500 | — |
| Cuscuta 4:1 See Extract | 100 | — |
| Tribulus Terrestris Vine | 375 | — |
| Butea Superba Root | 500 | — |
| Maca Root | 400 | — |
| Pine Bark extract | 300 | — |
| Cranberry extract | 80 | — |
| Avena Sativa extract seed | 60 | — |
| Sarsaparilla root | 80 | — |
| Swedish flower (pollen) | 60 | — |
| Pumpkin seed | 30 | — |
| Cayenne pepper | 800 | — |
| Vitamin E (ditocopheral acetate) | — | 60 |
| Zinc Aspartate (20% elemental Zinc) | 50 | — |
| L-Carnitine | 500 | — |
| L-Arginine HCL | 400 | — |
| L-Lysine | 250 | — |

As best shown in Table 3, the composition may include various ingredients in a range of effective amounts according to the disclosed method.

TABLE 3

| Component | mg | IU |
|---|---|---|
| Saw Palmetto extract | 100 | — |
| Damiana Leaf extract | 100 | — |
| Piper Nigrum Fruit Extract | 5 | — |
| Hawthorn Berry | 120 | — |
| Muira Paula Bark 4:1 extract | 75 | — |
| Korean Red Ginseng | 125 | — |
| Catuaba Bark 4:1 extract | 100 | — |
| Epimedium Saggatatum leaf | 55 | — |
| Ginkgo Biloba Leaf | 125 | — |
| Cuscuta 4:1 See Extract | 25 | — |
| Tribulus Terrestris Vine | 93.75 | — |
| Butea Superba Root | 125 | — |
| Maca Root | 100 | — |
| Pine Bark extract | 75 | — |
| Cranberry extract | 20 | — |
| Avena Sativa extract seed | 15 | — |
| Sarsaparilla root | 20 | — |
| Swedish flower (pollen) | 15 | — |
| Pumpkin seed | 7.5 | — |
| Cayenne pepper | 200 | — |
| Vitamin E (ditocopheral acetate) | — | 15 |
| Zinc Aspartate (20% elemental Zinc) | 12.5 | — |
| L-Carnitine | 125 | — |
| L-Arginine HCL | 100 | — |
| L-Lysine | 62.5 | — |

As best shown in Table 4, the composition may include various ingredients in a range of effective amounts according to the disclosed method.

TABLE 4

| Component | mg | IU | per serving | |
|---|---|---|---|---|
| Saw Palmetto extract | 375 | — | 47 | mg |
| Damiana Leaf extract | 400 | — | 100 | mg |
| Piper Nigrum Fruit Extract | 20 | — | 2.5 | mg |
| Hawthorn Berry | 420 | — | 52.5 | mg |
| Muira Paula Bark 4:1 extract | 300 | — | 38 | mg |
| Korean Red Ginseng | 425 | — | 54 | mg |
| Catuaba Bark 4:1 extract | 400 | — | 50 | mg |
| Epimedium Saggatatum leaf | 200 | — | 25 | mg |
| Ginkgo Biloba Leaf | 400 | — | 50 | mg |
| Cuscuta 4:1 See Extract | 100 | — | 12.5 | mg |
| Tribulus Terrestris Vine | 350 | — | 44 | mg |
| Butea Superba Root | 500 | — | 62.5 | mg |
| Maca Root | 400 | — | 50 | mg |
| Pine Bark extract | 300 | — | 38 | mg |
| Cranberry extract | 60 | — | 7.5 | mg |
| Avena Sativa extract seed | 50 | — | 6.25 | mg |
| Sarsaparilla root | 50 | — | 6.25 | mg |
| Swedish flower (pollen) | 50 | — | 6.25 | mg |
| Pumpkin seed | 30 | — | 3.75 | mg |
| Vitamin E (ditocopheral acetate) | — | 60 | 7.5 | IU |
| Zinc Aspartate (20% elemental | 30 | — | 3.75 | mg |
| Zinc) | | | | |
| L-Carnitine | 500 | — | 62.5 | mg |
| L-Arginine HCL | 300 | — | 38 | mg |
| L-Lysine | 250 | — | 32 | mg |
| Total | 5970 mg | | 749.75 | mg/capsule |

With respect to Table 4, these components or ingredients can be formulated into tablets or capsules and taken a specific number of times per day (e.g. eight times per day) to provide the maximum dose. Thus, each tablet or capsule will have ⅛ of the total daily amount listed. Each individual can begin with two daily capsules for one to two months. This can be increased to four tablets daily for one to two months and so on, until a maximum of eight tablets or capsules are taken daily.

According to an example study of a method of administering the disclosed composition, four participants ingested the disclosed composition once daily in the amount shown in Table 3 for approximately 3 months. Subsequently, the participants ingested the disclosed composition twice daily in the amount shown in Table 3 for approximately 3 months. For a period thereafter of approximately six months, participants were administered cannabis indica via inhalation daily in addition to ingested twice daily administration of the composition as shown in Table 3. Subsequently, for an additional six months, participants ingested the disclosed composition three times daily in the amount shown in Table 3 in addition to the administration of cannabis indica. Thereafter, participants ingested the disclosed composition four times daily in the amount shown in Table 3 in addition to ingesting 20 milligrams of tadalafil orally every five days in addition to the administration of cannabis indica. No participants were removed from the study due to medical issues including side effects. Heart rate and blood pressure remained in normal ranges throughout the duration of the study. Two participants experienced low grade nausea, particularly during the first 12 months of the study. Two participants reported occasional diarrhea. Two participants described occasional low-grade headaches and occasional low grade lightheadedness. No other side effects were reported.

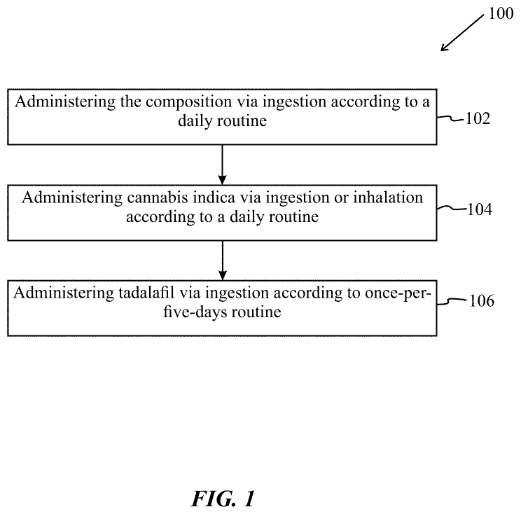

FIG. 1 depicts a simplified flowchart of a method 100 of administering the disclosed composition including step 102, administering the composition via ingestion by pill or capsule according to the previously described daily routine. According to some embodiments, ingestion of the composition may occur four times daily wherein the composition includes the components in the amount listed in Table 3. Step 104 may include administering cannabis indica via ingestion or inhalation according to a daily routine. According to some embodiments, ingestion or inhalation of cannabis indica may include inhaling the cannabis indica in the evening or prior to a participant going to sleep. Step 106 may include administering tadalafil via ingestion according to a routine of once per five days.

Participants reported increased energy including increased energy to a significant degree. One participant reported increased energy to a profound degree. All participants indicated that improved energy included no increased nervousness, anxiety, or jitteriness. All participants indicated significant improvement in mental clarity, mental alertness, and memory. Two participants reported significant improvement in general well-being and contentment, and two participants reported profound improvement in general well-being and contentment.

Sexual enhancement was measured according to numerous variables including anticipation prior to sexual activity, erectile strength or rigidity, pleasure or enjoyment during sexual activity including up to and just prior to climax or orgasm, intensity of each orgasm, duration of each orgasm, number of orgasms having a refractory period thereafter, estimations on refractory periods, number of orgasms without a subsequent refractory period, and time between orgasms.

Prior to the study of a method of administering the disclosed composition, participants reported their current sexual situation as “good to very good.” Prior to onset of the study, participants one and two reported sexual encounters two to three times per week participant three listed three to four sexual encounters per week. Participant four reported four to five sexual encounters per week.

After the initial three-month period described previously, all participants noted “mild but definite” improvement in the categories of anticipation prior to sexual activity, erectile strength or rigidity, pleasure or enjoyment during sexual activity including up to and just prior to climax or orgasm, intensity of each orgasm, and duration of each orgasm.

After the ingestion and twice daily administration of the composition as shown in Table 3 and the introduction of the inhalation of cannabis indica, participants reported “good to very good” improvement in the categories of anticipation prior to sexual activity, erectile strength or rigidity, pleasure or enjoyment during sexual activity including up to and just prior to climax or orgasm, intensity of each orgasm, and duration of each orgasm.

After participants increased the ingestion of the disclosed composition to three times daily in the amount shown in Table 3, all participants reported “very good to excellent” improvement in the categories of anticipation prior to sexual activity, erectile strength or rigidity, pleasure or enjoyment during sexual activity including up to and just prior to climax or orgasm, intensity of each orgasm, and duration of each orgasm. Two participants reported an increase in number of orgasms having a refractory period thereafter.

Subsequently, after introducing the ingestion of 20 mg of tadalafil each fifth day into the method, all participants noted “excellent to profound” improvement in the categories of anticipation prior to sexual activity, erectile strength or rigidity, pleasure or enjoyment during sexual activity including up to and just prior to climax or orgasm, intensity of each orgasm, and duration of each orgasm.

After participants increased the ingestion of the disclosed composition to four times daily in the amount shown in Table 3, all participants reported “excellent to profound” improvement in in the categories of anticipation prior to sexual activity, erectile strength or rigidity, pleasure or enjoyment during sexual activity including up to and just prior to climax or orgasm, intensity of each orgasm, duration of each orgasm, and number of orgasms having a refractory period thereafter.

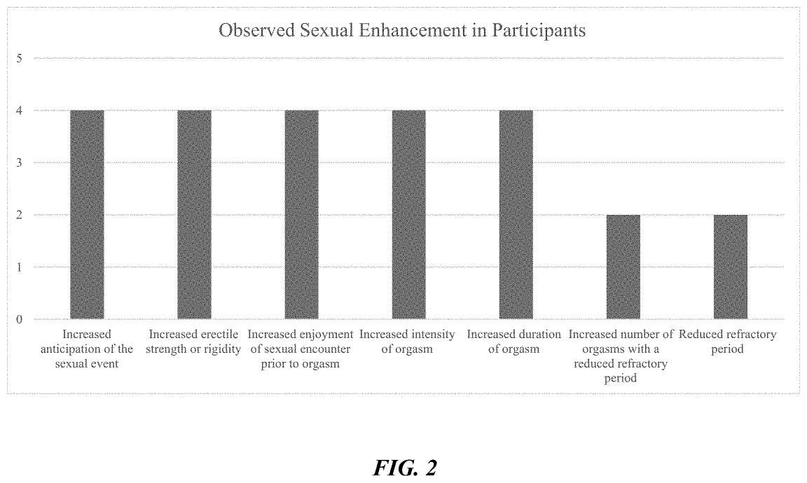

FIG. 2. depicts the previously described results including reported sexual enhancement for all participants in the categories of anticipation prior to sexual activity, erectile strength or rigidity, pleasure or enjoyment during sexual activity including up to and just prior to climax or orgasm, intensity of each orgasm, duration of each orgasm, number of orgasms having a refractory period thereafter, estimations on refractory periods, number of orgasms without a subsequent refractory period, and time between orgasms.

The following description of variants is only illustrative of components, elements, acts, product, and methods considered to be within the scope of the invention and are not in any way intended to limit such scope by what is specifically disclosed or not expressly set forth. The components, elements, acts, product, and methods as described herein may be combined and rearranged other than as expressly described herein and still are considered to be within the scope of the invention.

According to variation 1, a method of sexual enhancement may include administering a composition daily, the composition including saw palmetto extract; damiana leaf extract; Piper nigrum fruit extract; hawthorn berry; muira paula bark 4:1 extract; Korean red ginseng; Catuaba bark 4:1 extract; Epimedium sagittatum leaf; Ginkgo biloba leaf; cuscata 4:1 seed extract; Tribulus terrestris vine; butea superba root; maca root; pine bark extract; Cranberry extract; Avena sativa extract seed; sarsaparilla root; swedish flower; pumpkin seed; cayenne pepper; di-tocopheral acetate; zinc aspartate; L-carnitine; L-arginine HCl; and L-lysine. The method may further include administering cannabis indica daily and administering 20 mg of tadalafil once every five days.

According to variation 2, a composition for sexual enhancement may include saw palmetto extract; damiana leaf extract; Piper nigrum fruit extract; hawthorn berry; muira paula bark 4:1 extract; Korean red ginseng; Catuaba bark 4:1 extract; Epimedium sagittatum leaf; Ginkgo biloba leaf; cuscata 4:1 seed extract; Tribulus terrestris vine; butea superba root; maca root; pine bark extract; cranberry extract; Avena sativa extract seed; sarsaparilla root; swedish flower; pumpkin seed; cayenne pepper; di-tocopheral acetate; zinc aspartate; L-carnitine; L-arginine HCl; and L-lysine.

As used herein and in the appended claims, the singular forms “a”, “an”, and “the” include plural referents unless the context clearly dictates otherwise.

To enable the reader to obtain a clear understanding of the technological concepts described herein, the processes described herein generally include specific steps performed in a specific order. However, one or more of the steps of a particular process may be rearranged and/or omitted while remaining within the contemplated scope of the technology disclosed herein. One or more processes and/or steps thereof, may be combined, recombined, rearranged, omitted, or executed in parallel to create different process flows that are within the contemplated scope of the technology disclosed herein. While the processes herein may omit or briefly summarize some of the details of the technologies disclosed herein for clarity, the details described herein may be combined with process steps described herein to get a more complete and comprehensive understanding of these processes and the technologies disclosed herein.

In this disclosure, the descriptions of the various embodiments have been presented for purposes of illustration and are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein. Thus, the appended claims should be construed broadly, to include other variants and embodiments, which may be made by those skilled in the art.

It should be noted that all features, elements, components, functions, and steps described with respect to any embodiment provided herein are intended to be freely combinable and substitutable with those from any other embodiment. If a certain feature, element, component, function, or step is described with respect to only one embodiment, then it should be understood that that feature, element, component, function, or step can be used with every other embodiment described herein unless explicitly stated otherwise. This paragraph therefore serves as antecedent basis and written support for the introduction of claims, at any time, that combine features, elements, components, functions, and steps from different embodiments, or that substitute features, elements, components, functions, and steps from one embodiment with those of another, even if the following description does not explicitly state, in a particular instance, that such combinations or substitutions are possible. It is explicitly acknowledged that express recitation of every possible combination and substitution is overly burdensome, especially given that the permissibility of each and every such combination and substitution will be readily recognized by those of ordinary skill in the art.

I claim:

1. A method of sexual enhancement, comprising:
administering a composition daily, the composition comprising:
saw palmetto extract;
damiana leaf extract;
Piper nigrum fruit extract;
hawthorn berry;
muira paula bark 4:1 extract;
Korean red ginseng;
Catuaba bark 4:1 extract;
Epimedium sagittatum leaf;
Ginkgo biloba leaf;
cuscata 4:1 seed extract;
Tribulus terrestris vine;
butea superba root;
maca root;
pine bark extract;
cranberry extract;
Avena sativa extract seed;
sarsaparilla root;
swedish flower;
pumpkin seed;
cayenne pepper;
di-tocopheral acetate;
zinc aspartate;
L-carnitine;
L-arginine HCl; and
L-lysine.

2. A composition for sexual enhancement, comprising:
saw palmetto extract;
damiana leaf extract;
Piper nigrum fruit extract;
hawthorn berry;
muira paula bark 4:1 extract;
Korean red ginseng;
Catuaba bark 4:1 extract;
Epimedium sagittatum leaf;
Ginkgo biloba leaf;
cuscata 4:1 seed extract;
Tribulus terrestris vine;
butea superba root;
maca root;
pine bark extract;
cranberry extract;
Avena sativa extract seed;
sarsaparilla root;
swedish flower;
pumpkin seed;
cayenne pepper;
di-tocopheral acetate;
zinc aspartate;
L-carnitine;
L-arginine HCl; and
L-lysine.
